(19) United States
(12) Patent Application Publication
(10) Pub. No.: US 2004/0033271 A1
(43) Pub. Date: Feb. 19, 2004

(54) Methods for contemporaneous administration of levamisole and 5-fluorouracil

(76) Inventor: Seth Lederman, New York, NY (US)

Correspondence Address: Morgan & Finnegan, Suite 400, 1775 Eye Street, Washington, DC, 20006, US

(21) Appl. No.: 10/450,131

(22) PCT Filed: Dec. 3, 2001
(86) PCT No.: PCT/US01/46381

(51) Int. Cl. A61K 31/7072; A61K 31/66; A61K 31/525; A61K 31/337; A61K 33/24

(52) U.S. Cl. 424/649; 514/50; 514/34; 514/109; 514/449; 514/251

(57) ABSTRACT

The invention provides an improved method for potentiating the antineoplastic activity of 5-fluorouracil with levamisole, or with an analogue thereof. Specifically, the invention provides regimens wherein levamisole is administered contemporaneously with the administration of 5-fluorouracil, thereby increasing the exposure of tumor tissue to the simultaneous presence of the two drugs. The invention further provides methods for the parenteral administration of levamisole, thereby overcoming the disadvantages associated with inter-patient variability in the bioavailability of oral.

### FIELD OF THE INVENTION

This invention relates to the field of pharmaceuticals, and to the field of cancer chemotherapy in particular. Specifically, the invention relates to methods of contemporaneously co-administering the drug levamisole, or its analogues, with 5-fluorouracil or related drugs, in order to treat patients afflicted with cancer.

### BACKGROUND OF THE INVENTION

Levamisole was originally developed and marketed as a racemic mixture, as the anthelmintic drug tetramisole (D. Thienpont et al., 1966, Nature 209:1084). Most of this activity was subsequently found to reside in the levorotary isomer, levamisole (I):

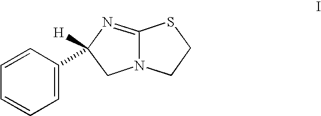

Anecdotal observations in animals and humans suggested that immune system function of immunologically compromised subjects might be improved by administration of levamisole. Numerous studies in the 1970s examined the effects of levamisole, in a variety of infectious, autoimmune, and oncologic diseases, with varying results. Although results were not consistent, patients with impaired phagocytic and lymphocytic function seemed to respond favorably to levamisole treatment, at least in some studies, while those with normal immune systems did not generally show an effect. In the course of these studies the anthelmintic regimen for the administration of levamisole was widely adopted, which was considered appropriate for obtaining the desired “immunomodulatory” effect. This protocol, referred to herein as the “immunomodulatory levamisole regimen,” called for a 50 mg oral dose of levamisole, taken three times a day, for three days. This three-day regimen was repeated every other week throughout the course of therapy, which typically ranged from six months to two years in length. This early work has been reviewed: P. Janssen, 1976, Progress Drug Res. 20:347-383; W. K. Amery, 1977, Cancer Treatment Reviews 4:167-194.

Because cancer has been viewed as a defect of immune surveillance, and because levamisole was thought to have immunomodulatory properties, some experimental work began to evaluate whether levamisole would be useful in stimulating the immune system of cancer patients. However, these studies of levamisole as a cancer chemotherapeutic agent proved inconclusive at best. Animal studies with Lewis lung 3LL tumors in mice were used to support claims to the use of levamisole in “aiding the regression of neoplastic disease” (U.S. Pat. No. 4,584,305), but these claims were not supported in subsequent human clinical trials of levamisole as a cancer monotherapy. Inverse dose-response effects were noted in at least two studies: effects that were seen at levamisole doses of 2-5 mg/kg were not observed at 10 mg/kg (D. Sampson et al., 1977, Cancer Res. 37:3526-3529; T. Hozumi, 1978, Gann 69:339-343). An increase in recurrence was observed in a large study of post-surgical treatment of breast cancer with levamisole (Exec. Committee Danish Breast Cancer Coop. Group, The Lancet, Oct. 18, 1980, 824-827). Human colorectal cancer xenografts in nude mice were shown to be unaffected by levamisole (Van Ginckel et al., 1992, Eur. J. Cancer 28A: 1137-11399).

Although some small-scale preliminary studies suggested that levamisole had a positive effect when administered as an adjuvant to surgery, the results of follow-up trials did not confirm the utility of levamisole alone as a chemotherapeutic drug in this context (A. Goldin et al, 1982, Recent Results Cancer Res. 80:351-356). For example, levamisole alone had no benefit in a study of Dukes' C. colon cancer (J.-P. Arnaud et al., 1989, Br. J. Surg. 76: 284-289). Similar results were obtained in one arm of a second, larger study (1296 patients) of Dukes' B and C colorectal cancer (C. Moertel, 1990, N. Engl. J. Med. 322:352-358). Janssen has acknowledged that levamisole “does not usually produce major benefit when given as monotherapy” (M. De Brabander et al., 1992, Anticancer Research 12:177-188).

In later studies, however, the superimposition of the immunomodulatory levamisole regimen onto other drug regimens demonstrated clinical benefits. Levamisole was shown to provide a significant improvement in overall survival and disease-free survival, and diminished cancer recurrence, when the immunomodulatory levamisole regimen was superimposed on a regimen of 5-fluorouracil (5-FU), after surgery for colorectal cancer (J. Laurie et al., 1989, J. Clinical Oncology, 7:1447-1456; C. Moertel, 1990, N. Engl. J. Med. 322:352-358). These studies have been reviewed (W. Amery et al., 1977, Cancer Treat. Reviews, 4:167-194; M. De Brabander et al., 1992, Anticancer Research 12:177-188; G. Masucci et al., 1991, Med. Oncol. & Tumor Pharmacother., 8:207-220). Although these studies found that the superimposition of the immunomodulatory levamisole regimen on a 5-FU regimen increased survival and decreased recurrence, the mechanism by which these results were obtained was not understood, and the effect was not optimized. As noted in a recent review, “no convincing mechanism of biologic synergy emerged” from the many studies of levamisole/5-FU adjuvant therapy (R. S. Midgley, D. J. Kerr, Hospital Practice, May 15, 2000, pp. 55-62). It should be noted that actual contemporaneous co-administration of levamisole and 5-FU in these studies was rare, being an incidental result arising from the superposition of the two different dosing schedules of the drugs. In fact, both drugs were rarely even administered on the same day. Even when administered on the same day, the timing of 5-FU administration was not prescribed to coincide with serum levamisole concentrations. As in the earlier studies of levamisole as monotherapy, “the dose and schedule of levamisole used in these studies was chosen arbitrarily from the anthelmintic experience.” (J. F. Cleary et al., 1997, Cancer Chemother. Pharmacol. 39:300-306):

Adjuvant therapy with the combination of 5-FU and levamisole became accepted as a standard post-surgical regimen for Dukes' C., stage III colon cancer patients in the United States (NIH Consensus Conference: Adjuvant therapy for patients with colon and rectal cancer, 1990, J. Am. Med. Assoc. 264:1444-1450). The dosing schedule for levamisole was adapted directly from the regimen used for anthelmintic treatment, and was selected on the basis that immunomodulation by levamisole was the desired effect. Because levamisole was already available in an oral tablet formulation, oral dosing was adopted as the standard method of administration. In addition, the standard immunomodulatory regimen of levamisole was used. Thus, a typical protocol is 50 mg levamisole, given orally every eight hours (t.i.d.) for three consecutive days (×3 days), the three-day administration being repeated every two weeks (q.o.w.). Superimposed on this schedule are five daily infusions of 5-FU (450 mg/m²) for the first five days, followed four weeks later by weekly infusions. This regimen is continued for one year (C. Moertel, 1995, Ann. Intern. Med. 122: 321-326). Under this protocol, administration of both drugs on the same day was a rare and incidental occurrence. Moreover, contemporaneous co-administration of the drugs, if it occurred at all, was a completely fortuitous occurrence.

The prior art dosing regimens did not contemplate that contemporaneous co-administration of levamisole and 5-FU would be necessary, or even have any advantage. This is because the regimens were designed with the assumption that a systemic immunomodulating activity was the central mechanism by which an immunomodulatory regimen of levamisole achieved the observed beneficial effects when superimposed upon a schedule of 5-FU administration. Under this assumption, oral levamisole, 50 mg t.i.d.×3 days, q.o.w., is presumed to create an altered immune state, in the context of which 5-FU is somehow able to function more effectively. Accordingly, prior art regimens consisted of independently designed and developed levamisole dose schedules and 5-FU dose schedules, which were then superimposed upon one another to arrive at the overall regimen. As discussed further below, the result is a regimen in which levamisole and 5-FU are in fact very rarely administered on the same day.

Interestingly, 5-FU regimens alone are ineffective against many cancers, including colorectal cancer. However, when superimposed on regimens of other drugs, 5-FU has been used with some success as a cancer chemotherapy, either primary or adjuvant, for anal cancer, colorectal cancer, biliary tract cancer, carcinoid tumors, cervical cancer, esophageal cancer, gastric cancer, head and neck cancer, hepatoblastoma, liver cancer, pancreatic cancer, prostate cancer, breast cancer and lung cancer.

Several mechanisms have been proposed for the anti-cancer effects of 5-FU. 5-FU inhibits thymidylate synthetase, an enzyme required for DNA synthesis, and tumor cells that exhibit high levels of thymidylate synthetase seem to be more sensitive to 5-FU, but there are other possible mechanisms of action. For example, 5-FU is preferentially metabolized in tumor cells into 5-FdUTP, a pseudobase that can be incorporated into DNA, and 5-FUTP which can be incorporated into RNA. 5-FU also induces apoptosis in cultured salivary gland culture cells by suppressing NF-κB activity (K. Aota et al., 2000, Biochem. Biophys. Res. Commun, 273:1168-1174). The relative contributions of these various activities to clinical cytotoxicity have not been worked out, and may well vary among different tumor types. Indeed, it has been suggested that the mechanism of action varies depending on the method of administration, with brief but high-dose exposure favoring incorporation of 5-FU into RNA, and extended lower-dose exposure favoring inhibition of thymidylate synthetase. Accordingly, it has been suggested that 5-FU should be considered as two different drugs, depending on the dosing regimen (A. Sobrero et al, 1997, J. Clin. Oncol. 15:368-381).

The mechanism by which the immunomodulatory regimen of levamisole exhibits synergy with the antineoplastic activity of 5-FU has not yet been determined. Some studies have examined whether pharmacological interactions between the two drugs may account for the clinical effects observed in trials where levamisole and 5-FU regimens have been superimposed. For example, one study examined whether levamisole and 5-FU interacted pharmacologically to inhibit growth in cultured tumor cell lines. In these studies no synergy could be observed at pharmacologically relevant concentrations, and the authors concluded that the data “do not indicate that a direct interaction of [levamisole] with 5-FU is a possible explanation for the therapeutic synergism observed.” (J. L. Grem, C. J. Allegra, 1989, J. N.C. I. 18:1413-1417).

In another example, the effects of levamisole and 5-FU were studied on the in vitro colony formation of tumor cell lines. At high doses of levamisole and 5-FU certain cell lines (A549 lung carcinoma cell line, A375 melanoma cell line) exhibited decreases in the number of colonies formed, but not others (COLO205 colon carcinoma cell line and MCF7 breast carcinoma cell line) did not. Although the relationship of this assay to clinical effects is uncertain, these studies showed that very high doses of levamisole decreased the number of colonies in the presence of 5-FU but not in the presence of the 5-FU metabolite 5-fluorodeoxyuridine. The most sensitive cell line in this assay, the A375 melanoma cell line, was affected by 30 μg/ml (0.125 μM) levamisole. These investigators suggested that levamisole may be able to inhibit intracellular phosphatases and thereby lead to an increased cellular retention of FdUMP (fluorodeoxyuridine monophosphate, the active metabolite of 5-FU). However, the concentrations of serum levamisole achieved in vivo upon oral administration are far lower than the concentrations that inhibit phosphatases (J. Kovach et al., 1990, Proc. Am. Assoc. Cancer Res. 31:399, abst. 2365).

Certain of the pharmacologic effects of levamisole are associated with compounds of the general structural class to which levamisole belongs, and are not specific to levamisole alone. For example, the 4-hydroxy and 4-bromo analogues of levamisole are also capable of inhibiting phosphatases (J. Kovach et al., 1992, J. Natl. Cancer Inst. 84:515-519). Dexamisole, the enantiomer of levamisole, is less active in potentiating the antineoplastic effect of BCNU against bladder cancer in rats (T. Hozumi, 1978, Gann 69:339-343). These observations suggest a selective interaction of levamisole and its analogues with one or more target proteins.

The bioavailability of oral levamisole is highly variable among individual human subjects. Luyckx et al. first noted this phenomenon (M. Luyckx et al., 1982, Eur. J. Drug Metab. Pharmacokinet. 7:247-254), and it has been confirmed in recent studies (P. Gwilt et al., 2000, Cancer Chemother. Pharmacol. 45:247-251). The latter authors observed a seven-fold difference between the highest and lowest AUC (area under curve) among twenty cancer patients receiving oral levamisole 50 mg, t.i.d. A five-fold increase in the recommended oral levamisole dose induces severe nausea and vomiting (J. Reid et al., 1998, Cancer Chemother. Pharmacol. 41:477-484), which would limit compliance with the regimen; meanwhile, of course, the lowered plasma level obtained in other patients is expected to result in a diminished degree of 5-FU potentiation.

Despite the potentially limiting problem of variable bioavailability associated with oral dosing, the administration of levamisole to humans by a parenteral route has not previously been reported. In large part, this is likely to be a consequence of the fact that, at the time the above-described adjuvant therapies for cancer were being developed, levamisole was readily available in tablet form in the approved and appropriate doses for oral administration to humans. IV administration has of course been a means of introducing levamisole to animals in the laboratory, but this route of administration has been associated with cardiac side-effects in at least two species: in rats cardiotoxicity was observed at IV doses as low as 2 mg/kg (G. Onuaguluchi, I. Igbo, 1990, Arch. Int. Pharmacodyn. 305:55-62), and ECG irregularities were seen in guinea pigs at 10 mg/kg IV (G. Onuaguluchi, I. Igbo, 1990, Afr. J. Med. Sci. 19:307-312). The effects are seen within 60 seconds of administration, and appear to be associated with the high transient concentrations that accompany a bolus injection.

To the knowledge of the present inventor, parenteral administration of levamisole contemporaneously with parenteral 5-FU has not been considered or even conceived by those skilled in the art. This may be due to a combination of factors, which includes the cardiotoxicity associated with bolus IV administration in some species, the convenience and availability of oral dose formulations, and the cost and inconvenience of preparing an IV infusion in the absence of a commercially available parenteral formulation. The notion that high hepatic concentrations of levamisole, generated by rapid oral absorption, are needed for activity would also discourage attempts at parenteral administration.

Partly for the reasons discussed above, adjuvant treatment of cancer with levamisole and 5-FU suffers from limited efficacy. Levamisole/5FU adjuvant therapy has been almost entirely supplanted in the United States by the combination of 5-FU with leucovorin (folinic acid) (M. O'Connell et al., 1997, Clin. Oncol. 15:246), and in fact distribution of the Ergamisol™ brand of levamisole, the only form available in the United States was recently discontinued by the manufacturer (Letter to physicians from Janssen Pharmaceutica, Oct. 5, 2000).

It is clear that levamisole and leucovorin operate by different mechanisms (Z.-G. Zhang and Y. M. Rustum, 1992, Sem. Oncol. 19(Suppl. 4):46-50; C. P. Spears et al., 1984, Cancer Res. 44:4144-50), thus, although leucovorin is a substitute for levamisole in the therapeutic sense it is not equivalent in a mechanistic sense. Levamisole and leucovorin are both effective as potentiators of 5-FU in adjuvant therapy, at least for Dukes' B or C colorectal cancer (N. Wolmark et al., 1999, J. Clin. Oncol. 17:3553-3559), therefore any improvement in the method of co-administration of levamisole and 5-FU would provide a superior drug regimen for adjuvant chemotherapy in the treatment of these conditions. Given the different mechanisms by which levamisole and leucovorin enable 5-FU to exhibit clinical effects, these are clearly two different therapies, and an improved method of co-administration of levamisole and 5-FU would also provide an alternative therapy for patients who do not respond adequately to leucovorin/5-FU therapy. A 5-FU regimen in which parenteral levamisole and leucovorin are alternately administered, or co-administered, should also be advantageous.

In view of the observed correlation between response to 5-FU/leucovorin adjuvant therapy and Bcl-2 overexpression (J. McKay et al., 2000, Int. J. Oncol. 17:153-158), it is also anticipated that potentiation of 5-FU with levamisole would offer some benefit when administered prophylactically to a subject exhibiting Bcl-2 overexpression in a pre-cancerous biopsy specimen, or exhibiting any other chromosomal marker or altered gene which might be found to correlate with outcome of adjuvant chemotherapy.

More recently, irinotecan (a topoisomerase I inhibitor) has been shown to be a useful salvage therapy for leucovorin/5-FU treatment failures. In a recent study irinotecan was administered in conjunction with leucovorin-5-FU therapy, and some benefit of triple therapy was demonstrated (L. Saltz et al., 2000, N. Engl. J. Med. 343:905-914). Ethynyluracil (eniluracil) also potentiates the cytotoxic effects of 5-FU, by inhibiting dihydropyrimidine phosphate dehydrogenase (D Baccanari et al., 1993, Proc. Natl. Acad. Sci. USA. 90:11064-11068). Addition of levamisole to any of these drug combinations is expected to provide a beneficial effect.

The drug capecitabine (Xeloda™) is an orally available prodrug of 5-FU that is activated by enzymes that are particularly prevalent in liver and tumor cells. For this reason it may be given in higher doses than 5-FU itself, and is believed to produce higher concentrations of 5-FU in the tumor tissue. It is currently approved for treatment of refractory breast cancer. Preclinical studies show that capecitabine has significant activity against a variety of tumor types when used as monotherapy and in combination with other chemotherapeutic agents (R. Schilsky, 2000, Oncology (Huntingt.) 14:1297-1306; 1309-1311). As capecitabine is a 5-FU prodrug, it is anticipated that levamisole would potentiate the activity of capecitabine, or of various other analogues or prodrugs of 5-FU that are currently in development, such as for example doxifluridine and ftorafir (tegafur).

Among the approved uses for 5-FU is as a component of the “CMF” (cyclophosphamide, methotrexate, and fluorouracil), “CEF” (cyclophosphamide, epirubicin, and fluorouracil), and “CAF” (cyclophosphamide, Adriamycin™ (doxorubicin), and fluorouracil) adjuvant chemotherapy regimes for breast cancer. An oral levamisole regimen has been superimposed on the CAF regimen in one trial with modest effects over untreated historical controls and no advantage over historical BCG immunotherapy controls (G. Hortobagyi et al., 1979, Cancer 43:1112-1122). The benefits of added levamisole obtained by Hortobayagi et al. were marginal and not widely accepted; it is the belief of the present inventor that this is due to the fact that there was no co-administration of 5-FU and levamisole at all in Hortobayagi's study. For the reasons set forth below, contemporaneous administration of levamisole can be expected to be of greater benefit in the CAP and CEF regimens.

The only example of levamisole and 5-FU being consistently simultaneously present in humans appears to have occurred in a pharmacokinetic and toxicologic investigation of high-dose levamisole (Reid et al., 1998, Cancer Chemother. Pharmacol. 41:477-484). In this study patients with advanced, inoperable cancers were dosed with 5-FU daily for five days, and levamisole was given orally 3 times a day for the same five days, with the morning dose given prior to the 5-FU injection. This was repeated every five weeks, with increasing doses of levamisole, up to doses of 300 mg/m² t.i.d. The maximum tolerable dose was found to be 100 mg/m² t.i.d. This study demonstrated that fairly high doses of levamisole can be given together with 5-FU. Since this was a toxicology study, and not an attempt to treat the patients' disease, Reid et al. made no attempt to statistically evaluate patient response to the treatment. A second pharmacokinetic study was recently reported (P. Gwilt et al., 2000, Cancer Chemother. Pharmacol. 45:247-251), in which adenocarcinoma patients were given 5-FU intravenously, followed by an oral dose of 50 mg levamisole. The usual immunomodulatory levamisole regimen (50 mg p.o., t.i.d. for 3 days) ensued. Given the 13 minute half-life of 5-FU (R. J. Fraile et al., 1980, Cancer Res. 40:2223-2228), and the 2-hour time to peak plasma concentration after oral levamisole administration, it is clear that the two drugs were not simultaneously present to any significant degree in Gwilt's study. The patients were not monitored beyond the last blood sample, 8 hours after the last dose of levamisole, thus no clinical responses were reported.

There remains a need for improved adjuvant chemotherapy, and primary chemotherapy, for the treatment of breast, colorectal, and other cancers, particularly adenomatous cancers. In particular, the problems of variable bioavailability and modest efficacy associated with the current schedule of oral administration of levamisole indicate a need for improved methods of obtaining synergy between 5-fluorouracil and levamisole.

### SUMMARY OF THE INVENTION

The present invention is based on the insight that, when present simultaneously in the body, levamisole and 5-FU interact to provide a chemotherapeutic effect. The effect is not due to a systemic immunomodulatory effect of levamisole, which was the basis for the design of prior art regimens, but is in fact due to a specific pharmacological interaction of levamisole with one or more of the molecular mechanisms surrounding the action of 5-FU. This leads to the further insight that it is advantageous for levamisole and 5-FU to be present simultaneously, at effective concentrations, in the patient. Without wishing to be bound by theory, it is believed that these agents can act simultaneously on the tumor cells that are the target of the therapy, to provide an antineoplastic effect that neither agent acting alone can produce. Accordingly, it is an object of the invention to provide novel regimens and methods for co-administration of levamisole and 5-FU, which ensure that the two drugs are simultaneously available at the site of action, at effective concentrations.

In general terms, the invention provides improved methods for potentiating the antineoplastic activity of 5-fluorouracil or its analogues with levamisole, or with analogues thereof. Specifically, the methods of the invention provide for the consistent contemporaneous administration of levamisole or its analogues with 5-fluorouracil or its analogues, thereby regularly exposing tumor tissue to effective concentrations of levamisole or an analogue at the same time that the tissue is being exposed to 5-FU or an analogue.

It is believed that the subset of cancer patients who do not respond well to the prior art adjuvant therapy regimen of oral levamisole and parenteral 5-FU very likely includes the many patients who exhibit poor bioavailability of levamisole after oral dosing. Accordingly, a further aspect of the present invention is a method for parenteral administration of levamisole in conjunction with 5-FU, which enables the consistent attainment of effective blood levels of levamisole in most patients.

The methods of the invention may optionally be employed in conjunction with the administration of other antitumor agents as well. The methods of the invention are expected to provide superior clinical outcomes to the prior art methods of administering levamisole in conjunction with 5-FU.

### DETAILED DESCRIPTION OF THE INVENTION

A dosage regimen has not heretofore been described in which 5-FU and levamisole are regularly co-administered. For example, the commonly used “Moertel” schedule (J. A. Laurie et al., 1989, J. Clin. Onc. 7:1447-1456; C. Moertel, 1995, Ann. Intern. Med. 122: 321-326; C. Moertel et al., 1990, N. Engl. J. Med. 322:352-358) of dosing 5-FU and levamisole can be represented as in Table 1.

TABLE 1
Moertel Protocol

| | Induction | | | | Maintenance | | | | |
|---|---|---|---|---|---|---|---|---|---|
| Week | 1 | 2 | 3 | 4 | 5 | 6 | 7 | 8 | 9-52 |
| Days | 1-7 | 8-14 | 15-21 | 22-28 | 29-35 | 36-42 | 43-49 | 50-57 | 58-364 |
| 5-FU | FFFFF** | ******* | ******* | ******* | F****** | F****** | F****** | F****** | q.w. |
| levamisole | LLL**** | ******* | LLL**** | ******* | LLL**** | ******* | LLL**** | ******* | q.o.w. |

(F: 5-fluorouracil dose given that day; L: levamisole (50 mg t.i.d.) given that day; *: no drugs that day)

It can be seen that on average, levamisole and 5-FU are actually administered on the same day only once every fourteen days in the course of therapy, while on three days out of every fourteen only one of the two drugs is given. The serum half-life of levamisole in humans is about four hours (J. M. Reid et al, 1998, Cancer Chemother. Pharmacol. 41:477-484; P. Gwilt et al., 2000, Cancer Chemother. Pharmacol. 45:247-251), while the serum half-life of 5-FU is only about 13 minutes (R. J. Fraile et al., 1980, Cancer Res. 40:2223-2228). Clearly, given the short half-lives of both drugs, the prior art regimen is remarkably far from optimal if, as the present inventor believes, the two drugs need to be present simultaneously for the desired clinical therapeutic effect to be obtained. Indeed, the two drugs are present together at significant concentrations in the serum only if oral levamisole is given shortly before the 5-FU infusion. Table 2 illustrates the percentages of treatment days and total days on which one or both drugs are administered.

TABLE 2
Co-administration under Moertel Protocol

| | Moertel Protocol | | | | | | | | |
|---|---|---|---|---|---|---|---|---|---|
| | Days on which one or both drugs are administered | | | | | | | | |
| | Induction | | | Maintenance | | | Total | | |
| | (28 days) | | | (336 days) | | | (364 days) | | |
| | No. | % of | | No. | % of | | No. | % of | |
| | of | treatment | total | of | treatment | total | of | treatment | total |
| | days | days | days | days | days | days | days | days | days |
| 5-FU only | 2 | 25 | 7 | 24 | 25 | 7 | 26 | 25 | 7 |
| Levamisole only | 3 | 38 | 11 | 48 | 50 | 14 | 51 | 49 | 14 |
| Levamisole + 5-FU | 3 | 38 | 11 | 24 | 25 | 7 | 27 | 26 | 7 |

The Moertel study found a 33% reduction in the death rate, and 16% improvement in 3½ year survival, associated with the superimposition of the immunomodulatory levamisole regimen upon the 5-FU regimen.

The QUASAR Study had two different schedules for 5-FU administration (QUASAR Collaborative Group, 2000, Lancet 355:1588-1596). One regimen was termed the “4-weekly schedule,” which involved six cycles of 4-week regimens. In the “4-weekly schedule,” incidental administration of 5-FU and levamisole on the same day occurred on three days, levamisole was administered alone on three days, and 5-FU was administered alone on two days (Table 3). The extent of co-administration of the drugs under this protocol is presented in Table 4.

TABLE 3
QUASAR 4-Weekly Protocol

| Week | 1 | 2 | 3 | 4 | 5-24 |
|---|---|---|---|---|---|
| Days | 1-7 | 8-14 | 15-21 | 22-28 | 29-168 |
| 5-FU | FFFFF** | ******* | ******* | ******* | q.d.x5 |
| Levamisole | LLL**** | ******* | LLL**** | ******* | q.o.w. |

(F: 5-fluorouracil dose given that day; L: levamisole (50 mg t.i.d.) given that day; *: no drugs that day)

TABLE 4
Co-administration under QUASAR 4-Weekly Protocol

| | Days on which one or both drugs are | | |
|---|---|---|---|
| | administered | | |
| | Weeks 1-24 (168 days) | | |
| | | % of | |
| | No. of | treatment | total |
| | days | days | days |
| 5-FU only | 12 | 25 | 7 |
| Levamisole only | 18 | 37 | 11 |
| Levamisole + 5-FU | 18 | 37 | 11 |

The QUASAR study also provided subjects the option of a “once weekly schedule” if the above “4-weekly schedule” was impracticable. In the “once-weekly” schedule, 5-FU is given alone half the treatment days and incidentally given on the same day as levamisole half the treatment days. In the last 6 weeks of the study, 5-FU is given alone, whenever it is administered. Levamisole is given on the same day as 5-FU only once in every three days in which levamisole is administered. The “once weekly schedule” and percentage of days with co-administration are shown in Tables 5 and 6.

TABLE 5
QUASAR Once-Weekly Protocol

| Week | 1 | 2 | 3-24 | 25 | 26-30 |
|---|---|---|---|---|---|
| Days | 1-7 | 8-14 | 15-168 | 169-176 | 177-210 |
| 5-FU | P****** | P****** | q.w. | P****** | q.w. |
| Levamisole | LLL**** | ******* | q.o.w. | ******* | None |

(F: 5-fluorouracil dose given that day; L: levamisole (50 mg t.i.d.) given that day; *: no drugs that day)

TABLE 6
Co-administration under QUASAR Once-Weekly Protocol

| | Days on which one or both drugs are administered | | | | | | | | |
|---|---|---|---|---|---|---|---|---|---|
| | Weeks 1-24 | | | After 24 weeks | | | Total | | |
| | (168 days) | | | (42 days) | | | (210 days) | | |
| | No. | % of | | No. | % of | | No. | % of | |
| | of | treatment | total | of | treatment | total | of | treatment | total |
| | days | days | days | days | days | days | days | days | days |
| 5-FU only | 12 | 25 | 7 | 6 | 100 | 14 | 18 | 33 | 8 |
| Levamisole only | 24 | 50 | 14 | 0 | 0 | 0 | 24 | 44 | 10 |
| Levamisole + 5-FU | 12 | 25 | 7 | 0 | 0 | 0 | 12 | 22 | 5 |

The QUASAR study found no additive effect from addition of levamisole to the leucovorin/5-FU regimen of adjuvant therapy after surgical resection for colorectal cancer. No significant differences in efficacy were seen between the 4-weekly and once-weekly regimens (D. J. Kerr et al., 2000, Ann. Oncol. 11:947-955).

Wolmark et al. administered 6 courses of 8 week cycles (Wolmark et al., 1999, J. Clin. Onc. 17:3553). During the 6 week period of 5-FU treatment, both drags are incidentally administered on the same day half of the days that 5-FU is administered, 5-FU is given alone on the other half of the treatment days, and levamisole is given alone on 6 days out of the 9 total days that it is administered. During the two week 5-FU “rest”, levamisole is always given alone. The 8-week dosing cycle and percentage of days in which both drugs were administered are shown in Tables 7 and 8.

TABLE 7
Wolmark Protocol

| | 8-Week Schedule is repeated 6 times | | | | | | | | |
|---|---|---|---|---|---|---|---|---|---|
| | 5-FU weekly | | | | | | 5-FU rest. | | |
| Week | 1 | 2 | 3 | 4 | 5 | 6 | 7 | 8 | 9-48 |
| Days | 1-7 | 8-14 | 15-21 | 22-28 | 29-35 | 36-42 | 43-49 | 50-57 | 58-336 |
| 5-FU | P****** | P****** | P****** | P****** | P****** | P****** | ******* | ******* | x6 |
| Levamisole | LLL**** | ******* | LLL**** | ******* | LLL**** | ******* | LLL**** | ******* | q.o.w. |

(F: 5-fluorouracil dose given that day; L: levamisole (50 mg t.i.d.) given that day; *: no drugs that day)

TABLE 8
Co-administration under Wolmark Protocol

| | Days on which one or both drugs are administered | | | | | |
|---|---|---|---|---|---|---|
| | One cycle | | | Total | | |
| | (56 days) | | | (336 days) | | |
| | No. | % of | | No. | % of | |
| | of | treatment | total | of | treatment | total |
| | days | days | days | days | days | days |
| 5-FU only | 3 | 20 | 5 | 18 | 20 | 5 |
| Levamisole only | 9 | 60 | 16 | 54 | 60 | 16 |
| Levamisole + 5-FU | 3 | 20 | 5 | 18 | 20 | 5 |

In the study by Hortobagyi et al. (1979, Cancer 43:1112-1122), 5-FU and levamisole were never administered on the same day (Table 9).

TABLE 9
Hortobagyi Protocol

| | 21 day cycle of FAC | | | |
|---|---|---|---|---|
| Week | 1 | 2 | 3 | 5-104 |
| Days | 1-7 | 8-14 | 15-21 | 22-730 |
| 5-FU | P****** | P****** | ******* | Day 1, Day 8 |
| Levamisole | ******* | *LL**LL | **LL*** | Days 9, 10, 13, |
| | | | | 14, 17, 18 |

(F: 5-fluorouracil given that day; L: levamisole (100 mg/m² p.o. t.i.d.) given that day; *: no drugs that day) Note: doxorubicin, cyclophosphamide, methotrexate not shown.

It is apparent that a mechanistic synergy between the drugs was not appreciated in any of the prior art approaches, and that contemporaneous administration, if it did happen, was a fortuitous event. Since the present inventor believes that a direct mechanistic interaction between levamisole and 5-FU is an essential component of an optimal therapy, the inventor believes that these fortuitous co-administration events are largely responsible for the observed synergy between the two drugs, which results in the clinical efficacy of the combination as adjuvant therapy in colorectal cancer. The present invention is based on the notion that deliberately orchestrating such co-administration events will considerably improve the outcome of combination therapy with levamisole and 5-FU. To the knowledge of the present inventor, regular, simultaneous or contemporaneous administration of 5-FU and levamisole as an adjuvant to surgery has not previously been described.

The present invention is based on the re-examination of all of the clinical literature on levamisole, which has revealed that incidental co-administration is the means by which clinical effects were seen.

For example, an early study (R. Windle et al., 1987, Br. J. Surg. 74:569-572) introduced a protocol in which patients received intravenous 5-FU immediately following surgery (resection of colon cancer) and on the first two post-operative days, oral levamisole (150 mg/po qd) on the first three post-operative days, and oral 5-FU (without levamisole) once a week for six months thereafter.

TABLE 10
Windle Protocol

| | 5-FU weekly | | |
|---|---|---|---|
| Week | 1 | 2 | 3-24 |
| Days | 1-7 | 8-14 | 15-168 |
| 5-FU | FFF**** | Fₒ****** | Fₒ****** |
| Levamisole | *LLL*** | ******* | ******* |

(F: 5-FU dose given that day; Fₒ: 5-FU given orally that day; L: levamisole (50 mg t.i.d.) given that day; *: no drugs that day)

This protocol, with only two days in which drugs were both administered (out of four that either was given), had a substantial advantage over 5-FU alone in this study. Re-interpreting the results of the Windle study in light of the present invention suggests that short periods of co-administration account for the clinical therapeutic effect, since later studies with only incidental subsequent co-administration failed to improve on the efficacy found in the Windle study.

In contrast to the state of knowledge of levamisole's mechanism of action, the molecular mechanism by which folinic acid (leucovorin) potentiates the cytotoxicity of 5-FU is regarded as established, and accordingly the two regimens for administering 5-FU plus leucovorin (the “Mayo Clinic” protocol, M. Poon et al., 1991, J. Clin. Oncol. 9:1967-1972; and the “Roswell Park” protocol, N. Petrelli et al, 1989, J. Clin. Oncol. 7:1419-1426) both call for simultaneous administration of 5-FU and leucovorin.

It has been observed in treating metastatic breast cancer that concurrent administration of a combination of antineoplastic drugs provides no long-term survival benefit over sequential administration, even when an increased response frequency and duration of response is obtained from the concurrent therapy (R. Chlebowsky et al., 1979, Cancer Res. 39:4503-4506). Consequently, there has been little motivation to co-administer drugs in the absence of an established mechanistic relationship between drugs, such as exists in the case of the leukovorin/5-FU combination.

It is an object of this invention to provide a method for potentiating the antineoplastic activity of 5-fluorouracil, or prodrugs thereof, which comprises the regular contemporaneous or concurrent administration of levamisole or an analogue thereof (see, e.g., U.S. Pat. No. 3,274,209). The route of administration may for example be oral, intravenous, subcutaneous, intramuscular, or intraperitoneal injection or infusion, or injection or infusion directly into the portal vein. Preferably the route is intravenous, more preferably it is intravenous infusion.

It is a particular object of the invention to provide for the parenteral administration of levamisole or an analogue thereof to a human, as a means of potentiating the antineoplastic effects of 5-fluorouracil, or a prodrug thereof, in vivo. The drug administered by the method of the invention is preferably levamisole, and the drug whose activity is to be potentiated is preferably 5-fluorouracil or capecitabine. Where the drug to be potentiated is 5-fluorouracil, the levamisole or levamisole analogue is most preferably administered contemporaneously. “Contemporaneously” means that the drugs are administered within about 90 minutes of each another, preferably within about 30 minutes of each other, and in the case of parenteral administration of levamisole, preferably simultaneously or within 10 minutes of each other. It is preferable that levamisole be administered first. Other drugs may be administered in addition to 5-fluorouracil and levamisole, for example leucovorin and/or ethynyluracil may be administered as an additional chemotherapeutic component.

The preferred analogues of levamisole are 4-bromolevamisole and 4-hydroxylevamisole, and prodrugs and pharmaceutically acceptable salts thereof. Preferred salts are hydrochloride and hydrobromide salts. The levamisole or levamisole analogues may be dissolved or suspended in any pharmaceutically acceptable vehicle suitable for parenteral administration. Preferred vehicles are sterile water and sterile saline.

In one embodiment of the invention, a conventional dose of levamisole (50 mg) is administered parenterally along with a conventional dose (450 mg/m²) of 5-fluorouracil. In order to potentiate the activity of any active 5-FU metabolites that may remain in the tumor cells, additional conventional doses, preferably one or two doses, of levamisole may optionally be administered parenterally over the next 24 hours. The procedure is repeated for 5 to 7 days, and weekly thereafter.

In an alternative embodiment of the invention, the first daily dose of levamisole is larger than a conventional oral dose, for example between 75 and 200 mg, preferably between 100 and 150 mg. In these embodiments as well, additional daily doses of levamisole may optionally be administered.

In another preferred embodiment, the optional daily doses of levamisole are administered orally. This embodiment has the advantage of being more amenable to out-patient treatment, as the attendance of medical personnel are required only once in the course of the day, while the optimum plasma level of levamisole that is provided by parenteral administration is obtained at the time the 5-fluorouracil is circulating.

In yet another embodiment of the invention, leucovorin may be administered contemporaneously with levamisole and 5-fluorouracil. In another embodiment, orally or parenterally administered capecitabine is substituted for 5-fluorouracil.

In another embodiment of the invention, parenteral levamisole is administered contemporaneously with 5-FU in any one of the “CMF” (cyclophosphamide, methotrexate, and 5-fluorouracil), “CEF” (cyclophosphamide, epirubicin, and 5-fluorouracil), or “CAF” (cyclophosphamide, Adriamycin™, and 5fluorouracil) adjuvant chemotherapy regimes for breast cancer, or is administered contemporaneously with 5-FU for the treatment of any cancer for which 5-FU is used alone or in combination with other drugs. It is anticipated that contemporaneous administration of levamisole may permit 5-FU to be used to treat cancers that have not previously been treated successfully with 5-FU, for example prostate cancer.

The methods of the invention are not intended to be limited to post-surgical adjuvant therapy, and treatment could be initiated prior to surgical reduction of tumors. In particular, parenteral administration of levamisole should make it possible to commence adjuvant therapy immediately after colorectal surgery, as there is no need to wait 20 days for healing of the intestinal tract as is typically the case when using oral levamisole.

### EXAMPLE 1

Levamisole hydrochloride (50 mg) is dissolved in pH 7.5 buffered saline (5 ml), and the solution is sterilized by ultrafiltration. The resulting solution is administered intravenously, simultaneously with an intravenous infusion of 450 mg/m² of 5-fluorouracil, to a patient who has undergone surgical resection of a colorectal cancer the previous day. Levamisole (50 mg) is administered twice more, at eight-hour intervals, by injection. The process is repeated for five consecutive days, and weekly thereafter for one year.

### EXAMPLE 2

The process of Example 1 is carried out, except that the second and third daily doses of levamisole are given orally, and treatment is commenced 20 days after surgery.

### EXAMPLE 3

Levamisole hydrochloride (100 mg) is dissolved in pH 7.5 buffered saline (5 ml), and the solution is sterilized by ultrafiltration. The resulting solution is administered intravenously, simultaneously with an intravenous infusion of 450 mg/m² of 5-fluorouracil, to a patient who has undergone surgical resection of a colorectal cancer the previous day. Levamisole (50 mg) is administered twelve hours later, by injection. The process is repeated for five consecutive days, and weekly thereafter for one year.

### EXAMPLE 4

The process of Example 3 is carried out, except that the second daily doses of levamisole are given orally, and treatment is commenced 20 days after surgery.

### EXAMPLE 5

Levamisole hydrochloride (100 mg) is dissolved in pH 7.5 buffered saline (5 ml), and the solution is sterilized by ultrafiltration. The resulting solution is administered intravenously, simultaneously with an intravenous infusion of 450 mg/m² of 5-fluorouracil, to a patient who has undergone surgical resection of a colorectal cancer at least 20 days previously. The process is repeated for five consecutive days, and weekly thereafter for one year.

### EXAMPLE 6

Levamisole hydrochloride (100 mg) is dissolved in pH 7.5 buffered saline (5 ml), and the solution is sterilized by ultrafiltration. The resulting solution is administered intravenously, simultaneously with an intravenous infusion of 500 mg/m² of 5-fluorouracil, to a patient who has undergone surgical resection of a colorectal cancer at least 20 days previously. The process is repeated once weekly for six consecutive weeks, followed by three weeks without drugs. This nine-week cycle is repeated a total of four times.

### EXAMPLE 7

Levamisole hydrochloride (100 mg) is dissolved in pH 7.5 buffered saline (5 ml), and the solution is sterilized by ultrafiltration. The resulting solution is administered intravenously, simultaneously with an intravenous infusion of 450 mg/m² of 5-fluorouracil, to a patient who has undergone surgical resection of a colorectal cancer. The process is repeated for five consecutive days, followed by three weeks without drugs. This four-week cycle is repeated six times.

### EXAMPLE 8

Levamisole hydrochloride (90 mg) is administered orally to a patient who has undergone surgical resection for colorectal cancer 20 days previously. An intravenous infusion of 450 mg/m² of 5-fluorouracil is commenced two hours later. The process is repeated for five consecutive days, followed by three weeks without drugs. This four-week cycle is repeated six times.

### EXAMPLE 9

On day 1 of a 21-day course of chemotherapy, levamisole hydrochloride (90 mg) is administered orally, and within 2 hours intravenous infusions of 500 mg/m² of 5-fluorouracil, 50 mg/m² of adriamycin, and 500 mg/m² of cyclophosphamide are administered, to a patient who has metastatic breast cancer. The process is repeated on day 8. The 21-day course is repeated until a total of 450 mg/m² of adriamycin has been administered, and 30 mg/m² methotrexate i.m. is substituted for adriamycin thereafter. Treatment is continued until remission is achieved, and for two additional years beyond that. claims

I claim:

1. A method of potentiating, in a human subject, the antineoplastic effects of a first drug selected from the group consisting of 5-fluorouracil, analogues of 5-fluorouracil, and prodrugs of 5-fluorouracil; the method comprising contemporaneously administering a second drug selected from the group consisting of: levamisole, 4-bromolevamisole, and 4-hydroxylevamisole, and salts and pro-drugs thereof.

2. The method of claim 1, wherein the human subject is suffering from a disease selected from the group consisting of anal cancer, colorectal cancer, biliary tract cancer, carcinoid tumors, cervical cancer, esophageal cancer, gastric cancer, head and neck cancer, hepatoblastoma, liver cancer, breast cancer, pancreatic cancer, prostate cancer, and lung cancer.

3. The method of claim 2, wherein the human subject is suffering from colorectal cancer.

4. The method of claim 2, wherein the human subject is suffering from breast cancer.

5. The method of claim 1, wherein the first drug is 5-fluorouracil.

6. The method of claim 2, wherein the first drug is 5-fluorouracil.

7. The method of claim 3, wherein the first drug is 5-fluorouracil.

8. The method of claim 4, wherein the first drug is 5-fluorouracil.

9. The method of any one of claims 1 through 8, wherein the second drug is levamisole.

10. The method of any one of claims 1 through 8, wherein the method further comprises the administration of one or more drugs selected from the group consisting of: leucovorin, doxorubicin, cyclophosphamide, epirubicin, irinotecan, paclitaxel, docetaxel, cisplatin, methotrexate, and ethynyluracil.

11. The method of claim 9, wherein the method further comprises the administration of one or more drugs selected from the group consisting of: leucovorin, doxorubicin, cyclophosphamide, epirubicin, irinotecan, paclitaxel, docetaxel, cisplatin, methotrexate, and ethynyluracil.

12. A method of potentiating, in a human subject, the antineoplastic effects of a first drug selected from the group consisting of 5-fluorouracil, analogues of 5-fluorouracil, and prodrugs of 5-fluorouracil; the method comprising administering via a parenteral route a second drug selected from the group consisting of: levamisole, 4-bromolevamisole, and 4-hydroxylevamisole, and salts and pro-drugs thereof.

13. The method of claim 12, wherein the human subject is suffering from a disease selected from the group consisting of anal cancer, colorectal cancer, biliary tract cancer, carcinoid tumors, cervical cancer, esophageal cancer, gastric cancer, head and neck cancer, hepatoblastoma, liver cancer, breast cancer, pancreatic cancer, prostate cancer, and lung cancer.

14. The method of claim 13, wherein the human subject is suffering from colorectal cancer.

15. The method of claim 13, wherein the human subject is suffering from breast cancer.

16. The method of claim 12, wherein the first drug is 5-fluorouracil.

17. The method of claim 13, wherein the first drug is 5-fluorouracil.

18. The method of claim 14, wherein the first drug is 5-fluorouracil.

19. The method of claim 15, wherein the first drug is 5-fluorouracil.

20. The method of any one of claims 12 through 19, wherein the second drug is levamisole.

21. The method of any one of claims 12 through 19, wherein the method further comprises the administration of one or more drugs selected from the group consisting of: leucovorin, doxorubicin, cyclophosphamide, epirubicin, irinotecan, paclitaxel, docetaxel, cisplatin, methotrexate, and ethynyluracil.

22. The method of claim 20, wherein the method further comprises the administration of one or more drugs selected from the group consisting of: leucovorin, doxorubicin, cyclophosphamide, epirubicin, irinotecan, paclitaxel, docetaxel, cisplatin, methotrexate, and ethynyluracil.

23. A method of potentiating, in a human subject, the antineoplastic effects of a first drug selected from the group consisting of 5-fluorouracil, analogues of 5-fluorouracil, and prodrugs of 5-fluorouracil; the method comprising contemporaneously administering via a parenteral route a second drug selected from the group consisting of: levamisole, 4-bromolevamisole, and 4-hydroxylevamisole, and salts and pro-drugs thereof.

24. The method of claim 23, wherein the human subject is suffering from a disease selected from the group consisting of anal cancer, colorectal cancer, biliary tract cancer, carcinoid tumors, cervical cancer, esophageal cancer, gastric cancer, head and neck cancer, hepatoblastoma, liver cancer, breast cancer, pancreatic cancer, prostate cancer, and lung cancer.

25. The method of claim 24, wherein the human subject is suffering from colorectal cancer.

26. The method of claim 24, wherein the human subject is suffering from breast cancer.

27. The method of claim 23, wherein the first drug is 5-fluorouracil.

28. The method of claim 24, wherein the first drug is 5-fluorouracil.

29. The method of claim 25, wherein the first drug is 5-fluorouracil.

30. The method of claim 26, wherein the first drug is 5-fluorouracil.

31. The method of any one of claims 23 through 30, wherein the second drug is levamisole.

32. The method of any one of claims 23 through 30, wherein the method further comprises the administration of one or more drugs selected from the group consisting of: leucovorin, doxorubicin, cyclophosphamide, epirubicin, irinotecan, paclitaxel, docetaxel, cisplatin, methotrexate, and ethynyluracil.

33. The method of claim 31, wherein the method further comprises the administration of one or more drugs selected from the group consisting of: leucovorin, doxorubicin, cyclophosphamide, epirubicin, irinotecan, paclitaxel, docetaxel, cisplatin, methotrexate, and ethynyluracil.
